(19) United States
(12) Patent Application Publication
(10) Pub. No.: US 2025/0312501 A1
(43) Pub. Date: Oct. 9, 2025

(54) WEARABLE DEVICE AND METHOD OF USING THE SAME

(71) Applicant: Po-Kang LIN, Taipei City (TW)

(72) Inventor: Po-Kang LIN, Taipei City (TW)

(21) Appl. No.: 18/292,894

(22) PCT Filed: Aug. 17, 2021
(86) PCT No.: PCT/CN2021/112997

§ 371 Date: Oct. 11, 2024

(51) Int. Cl. A61L 2/10

(52) U.S. Cl. CPC A61L 2/10; A61L 2202/11; A61L 2202/16

(57) ABSTRACT

The present disclosure provides a wearable device, including: a spectacle frame including a left spectacle frame and a right spectacle frame, the left spectacle frame is connected to the right spectacle frame through a bridge; at least one ultraviolet light source provided on the left spectacle frame and/or the right spectacle frame; a left temple connected to the left spectacle frame; a right temple connected to the right spectacle frame; and a power supply device provided in the spectacle frame, the left temple, or the right temple, and the power supply device is electrically connected to the at least one ultraviolet light source, wherein the at least one ultraviolet light source emits ultraviolet light to a facial and surrounding area of a user. Furthermore, the present disclosure also provides a method of using the wearable device.

### FIELD OF THE INVENTION

The present disclosure relates to a wearable device and a method of using the same, and in particular to a wearable device including an ultraviolet light source and a method of using the same.

### THE PRIOR ARTS

In response to the current impact of the epidemic, people always wear glasses, goggles and/or masks to reduce person-to-person droplet infection, however, apart from reducing droplet infection by means of basic goggles and masks, there is currently no further methods for blocking droplets and bacteria, which will also cause the user to worry about the blocking ability of goggles or masks.

### SUMMARY OF THE INVENTION

The technical object to be achieved by the embodiments of the present disclosure is to provide a wearable device and a method of using the same to solve the problem that the development of the structure of current goggles or glasses has reached a certain bottleneck in blocking the transmission of droplets or bacteria, and the user may have concerns about the blocking ability and blocking effect of goggles or masks.

In order to solve the above technical problems, embodiments of the present disclosure provide a wearable device, comprising: a spectacle frame including a left spectacle frame and a right spectacle frame, the left spectacle frame is connected to the right spectacle frame through a bridge; at least one ultraviolet light source provided on the left spectacle frame and/or the right spectacle frame; a left temple connected to the left spectacle frame; a right temple connected to the right spectacle frame; and a power supply device provided in the spectacle frame, the left temple, or the right temple, and the power supply device is electrically connected to the at least one ultraviolet light source, wherein the at least one ultraviolet light source emits ultraviolet light to a facial and surrounding area of a user.

Specifically, the wearable device as described above further comprises a control module, the control module is provided in the spectacle frame, the left temple, or the right temple, and the control module is electrically connected to the power supply device and the at least one ultraviolet light source, the control module controls the at least one ultraviolet light source to irradiate at a preset frequency.

Optionally, in the wearable device as described above, the preset period is greater than or equal to 1/30 second and less than or equal to 1/15 second.

Optionally, in the wearable device as described above, the at least one ultraviolet light source is provided on a top surface of the left spectacle frame and/or a top surface of the right spectacle frame.

Specifically, in the wearable device as described above, there is an included angle between the at least one ultraviolet light source and the top surface of the left spectacle frame and/or the top surface of the right spectacle frame, such that the ultraviolet light source irradiates an oronasal and surrounding position in the facial and surrounding area of the user, wherein the included angle is greater than or equal to 10 degrees and less than or equal to 60 degrees.

Optionally, in the wearable device as described above, the at least one ultraviolet light source is provided at a lower left corner of the left spectacle frame and/or a lower right corner of the right spectacle frame.

Specifically, in the wearable device as described above, there is an included angle between the at least one ultraviolet light source and a left edge of the left spectacle frame and/or a right edge of the right spectacle frame, such that the ultraviolet light source irradiates an oronasal and surrounding position in the facial and surrounding area of the user, wherein the included angle is greater than or equal to 10 degrees and less than or equal to 60 degrees.

Further, in the wearable device as described above, further comprising a switch module, the switch module is provided on the spectacle frame, the left temple, or the right temple, and the switch module is electrically connected to the power supply device and the at least one ultraviolet light source, the switch module controls a switching of the at least one ultraviolet light source.

Optionally, in the wearable device as described above, the at least one ultraviolet light source is at least one deep ultraviolet light source.

Another optional embodiment of the present disclosure also provides a method of using a wearable device, applied to the wearable device as described above, in a case where the wearable device has a control module, the method comprises the following steps:
controlling the at least one ultraviolet light source to emit ultraviolet light to the facial and surrounding area of the user by the control module to disinfect and sterilize the facial and surrounding area of the user, wherein the wearable device is worn on ears of the user through the left temple and the right temple, and is fixed to a nose of the user through the bridge.

Optionally, in the method as described above, further comprising the following steps: controlling and adjusting an irradiation angle of the at least one ultraviolet light source by the control module, such that the at least one ultraviolet light source irradiates an oronasal and surrounding position in the facial and surrounding area of the user.

Specifically, in the method as described above, the control module controls the at least one ultraviolet light source to irradiate at a preset frequency.

Compared with related arts, the wearable device and the method of using the same provided by embodiments of the present disclosure have at least the following beneficial effects:

The wearable device of the present disclosure can emit ultraviolet light to the facial and surrounding area of the user, and disinfect and sterilize the facial and surrounding area of the user through the ultraviolet light. When the user has worn the mask and covered the nose and mouth with the mask, the wearable device can emit ultraviolet light to disinfect and sterilize the mask and the area in front of the mask, eliminate bacteria attached to the mask, improve the blocking effect, thereby allowing the user to rest assured. Therefore, the wearable device of the present disclosure not only can provide basic droplet blocking ability to protect the eye portion of the user, but also can further provide sterilization and disinfection effect on the facial and surrounding area of the user.

### BRIEF DESCRIPTION OF THE DRAWINGS

FIG. 1 is a schematic diagram of a structure of a wearable device according to an embodiment of the present disclosure.

FIG. 2 is a schematic diagram of a structure of a wearable device according to another embodiment of the present disclosure.

FIG. 3A is a schematic diagram of the wearable device according to an embodiment of the present disclosure when being worn viewed from the front side.

FIG. 3B is a schematic diagram of the wearable device according to an embodiment of the present disclosure when being worn viewed from the lateral side.

FIG. 4 is a schematic diagram of the wearable device according to another embodiment of the present disclosure when being worn viewed from the front side.

FIG. 5 is a schematic diagram of the wearable device according to yet another embodiment of the present disclosure when being worn viewed from the front side.

FIG. 6 is a schematic diagram of a structure of a wearable device according to yet another embodiment of the present disclosure.

FIG. 7 is a schematic flowchart of a method of using the wearable device according to an embodiment of the present disclosure.

### REFERENCE NUMERALS

1, 2, 3: wearable device
10: spectacle frame
101: left spectacle frame
103: right spectacle frame
105: bridge
107: top surface
20: ultraviolet light source
30: left temple
40: right temple
50: power supply device
60: user
601: ears
603: nose
605: mouth
70: mask
80: control module
90: switch module
A1: facial and surrounding area
d1, d2: included angle

### DETAILED DESCRIPTION OF THE PREFERRED EMBODIMENT

In order to make the technical problems to be solved, technical solutions and advantages of the present disclosure clearer, a detailed description will be given below with reference to the accompanying drawings and specific embodiments. In the following description, certain details, such as specific configurations and components, are provided solely to assist in a comprehensively understanding of embodiments of the present disclosure. Accordingly, it will be apparent to a person skilled in the art that various changes and modifications can be made to the embodiments described herein without departing from the scope and spirit of the present disclosure. Additionally, descriptions for known functions and construction are omitted for clarity and conciseness.

It should be understood that reference throughout the present specification to “one embodiment” or “an embodiment” means that particular features, structures, or characteristics associated with the embodiment are included in at least one embodiment of the present disclosure. Thus, the appearance of “in one embodiment” or “in an embodiment” in various paragraphs throughout the present specification does not necessarily refer to the same embodiment. Furthermore, these particular features, structures or characteristics may be combined in one or more embodiments in any suitable manner.

In various embodiments of the present disclosure, it should be understood that the serial numbers of the following processes does not mean the conducting order, the conducting order of each process should be determined by functions and internal logic thereof, and should not form any limitations to the implementation process of the embodiments of the present disclosure.

It should be understood that the term “and/or” used herein is merely a description for the relationship of the related objects, which indicates that there can be three types of relationships, e.g., the term “A and/or B” can mean three situations including: A existing alone, both A and B existing, and B existing alone. In addition, the character “/” used herein generally indicates that the preceding and following related objects are in an “or” relationship.

In the embodiments provided by the present disclosure, it should be understood that “B corresponding to A” means that B is associated with A, and B can be determined according to A. However, it should also be understood that determining B according to A does not mean determining B only according to A, and B can also be determined according to A and/or other information.

### First Embodiment

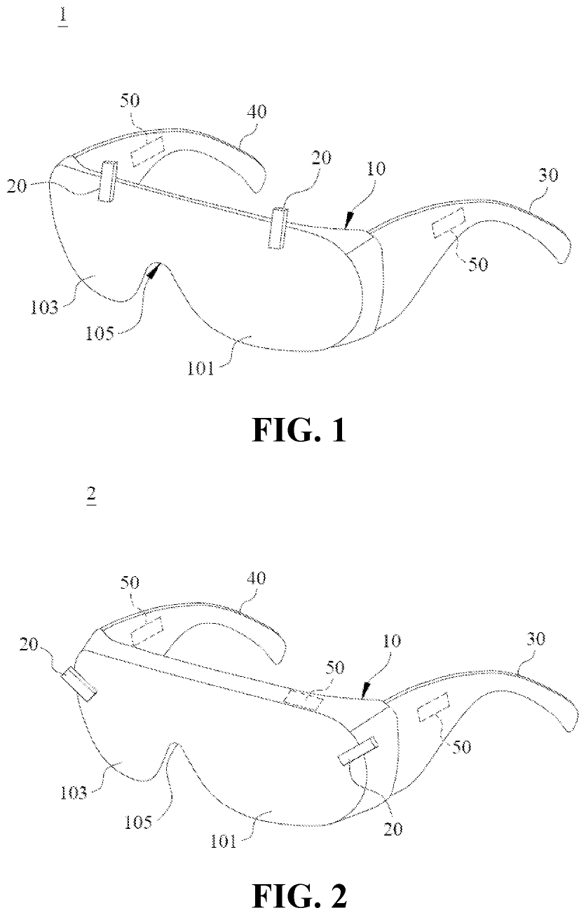

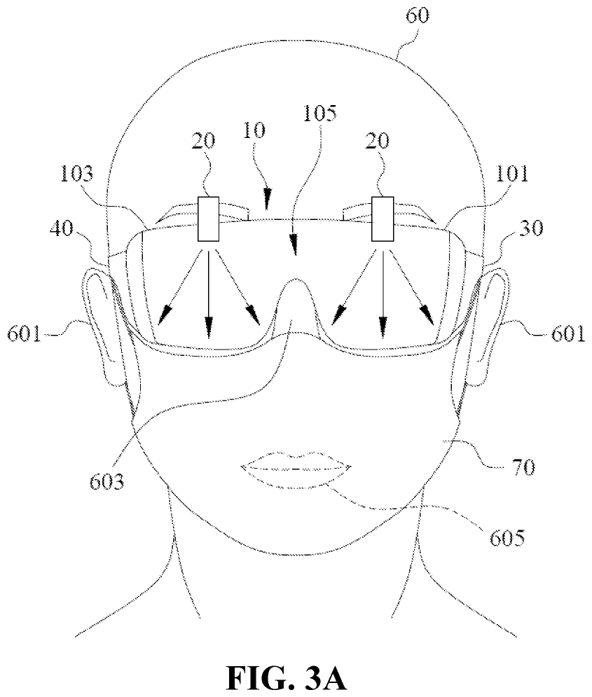

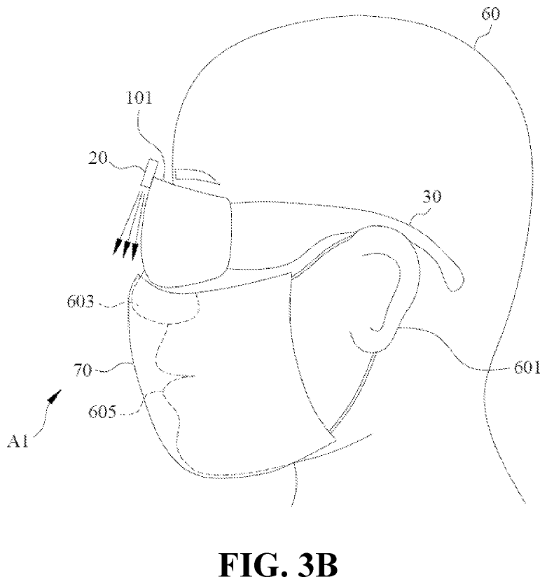

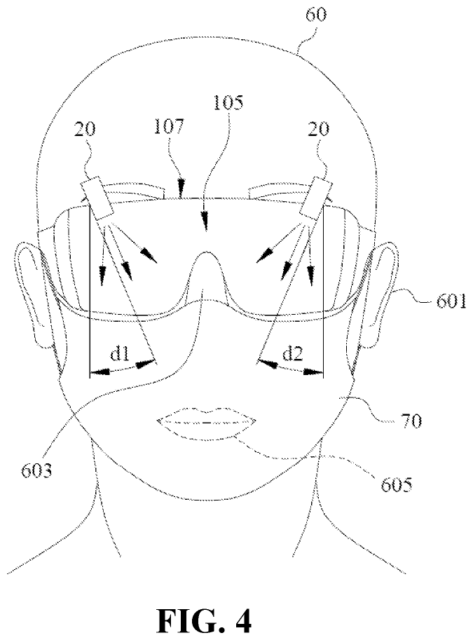

FIG. 1 is a schematic diagram illustrating a structure of a wearable device according to an embodiment of the present disclosure. Referring to FIG. 1, in an optional embodiment of the present disclosure, a wearable device 1 comprises: a spectacle frame 10 including a left spectacle frame 101 and a right spectacle frame 103, the left spectacle frame 101 is connected to the right spectacle frame 103 through a bridge 105; at least one ultraviolet light source 20 provided on the left spectacle frame 101 and/or the right spectacle frame 103, wherein in this embodiment, the ultraviolet light source 20 is provided on a top surface 107 of the left spectacle frame 101 and/or the right spectacle frame 103 (as shown in FIG. 4); a left temple 30 connected to the left spectacle frame 101; a right temple 40 connected to the right spectacle frame 103; and a power supply device 50 provided in the spectacle frame 10, the left temple 30, or the right temple 40, and the power supply device 50 is electrically connected to the at least one ultraviolet light source 20. Wherein the ultraviolet light source 20 emits ultraviolet light to a facial and surrounding area A1 of a user (as shown in FIG. 3B).

It should be noted that the ultraviolet light source 20 can be provided at any position on the left spectacle frame 101 and/or the right spectacle frame 103.

On the other hand, the at least one ultraviolet light source 20 can be provided on the left spectacle frame 101 and/or the right spectacle frame 103 by means of catching, hanging, clamping, adhesion or other arrangement. In addition, the ultraviolet light source 20 can be a deep ultraviolet light source (Ultraviolet-C, UVC), and the wavelength of the light emitted by the deep ultraviolet light source can be 100-300 nm, 280 nm, 275 nm or 274 nm, the ultraviolet light with these wavelengths has optimal disinfection and sterilization effect without harming the skin.

In addition, the ultraviolet light source 20 may be provided only on the left spectacle frame 101 or the right spectacle frame 103, or the ultraviolet light source 20 is provided on both the left spectacle frame 101 and the right spectacle frame 103. In addition, although FIG. 1 shows that two power supply devices 50 are provided, the power supply devices 50 can be selectively provided in the spectacle frame 10, the left temple 30, or the right temple 40, or provided according to the number of the ultraviolet light source(s) 20.

### Second Embodiment

FIG. 2 is a schematic diagram illustrating a structure of a wearable device according to another embodiment of the present disclosure. In this embodiment, the ultraviolet light source 20 of the wearable device 2 is provided on the left edge of the left spectacle frame 101 and/or the right edge of the right spectacle frame 103. Therefore, the disposing position of the ultraviolet light source 20 of the present disclosure can be adjusted according to actual usage conditions. In addition, since the spectacle frame 10 may have a certain thickness, the power supply device 50 may also be provided in the spectacle frame 10. It should be noted that although FIG. 2 shows that three power supply devices 50 are provided, the power supply devices 50 can be selectively provided in the spectacle frame 10, the left temple 30, or the right temple 40, or provided according to the number of the ultraviolet light source(s) 20.

### Third Embodiment

FIG. 3A is a schematic diagram illustrating the wearable device according to an embodiment of the present disclosure when being worn viewed from the front side; FIG. 3B is a schematic diagram illustrating the wearable device according to an embodiment of the present disclosure when being worn viewed from the lateral side. Referring to FIGS. 1, 3A and 3B, the wearable device 1 can be worn on ears 601 of a user 60 through the left temple 30 and the right temple 40, and the wearable device 1 is fixed to a nose 603 of the user 60 through the bridge 105. After the user 60 has finished wearing, the ultraviolet light source 20 emits ultraviolet light to the facial and surrounding area A1 of the user 60, and the facial and surrounding area A1 of the user 60 is disinfected and sterilized by the ultraviolet light emitted by the ultraviolet light source 20.

Further, when the user 60 has worn the mask 70 and covered the nose 603 and a mouth 605 with the mask 70, the ultraviolet light source 20 can emit ultraviolet light to disinfect and sterilize the mask 70, eliminate bacteria attached to the mask 70, thereby allowing the user 60 to rest assured. Therefore, the wearable device 1 of the present disclosure not only can provide basic droplet blocking ability, but also can further provide sterilization and disinfection effect on the facial and surrounding area A1 of the user 60. It should be noted that even if the user 60 does not wear the mask 70, the facial and surrounding area A1 of the user 60 still can be disinfected and sterilized by the ultraviolet light emitted by the ultraviolet light source 20.

### Fourth Embodiment

FIG. 4 is a schematic diagram illustrating the wearable device according to another embodiment of the present disclosure when being worn viewed from the front side. Referring to FIGS. 1 and 4, in this embodiment, there are an included angle d1 between one ultraviolet light source 20 and the top surface 107 of the right spectacle frame 103, and an included angle d2 between another ultraviolet light source 20 and the left spectacle frame 101. The included angle d1 and the included angle d2 can be any angle in the range of 10-60 degrees, such that the ultraviolet light source 20 can irradiate around, in front of, and above the oronasal position in the facial and surrounding area A1 of the user 60, so as to strengthen the disinfection and sterilization effect on the oronasal and surrounding position of the user 60.

It should be noted that in this embodiment, the ultraviolet light source 20 may be provided only on the top surface 107 of the left spectacle frame 101 or the top surface 107 of the right spectacle frame 103, or the ultraviolet light source 20 is provided on the top surface 107 of both the left spectacle frame 101 and the right spectacle frame 103.

### Fifth Embodiment

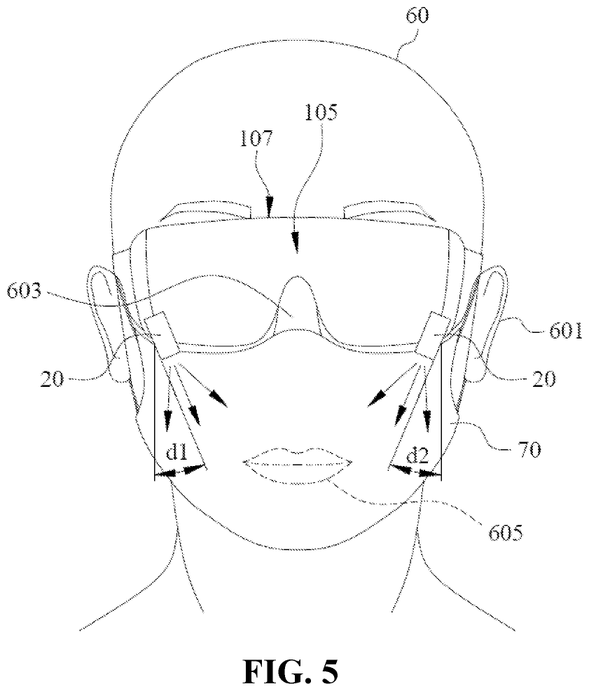

FIG. 5 is a schematic diagram illustrating the wearable device when being worn according to yet another embodiment of the present disclosure when being worn viewed from the front side. Referring to FIGS. 1 and 5, in this embodiment, the ultraviolet light sources 20 are provided at the lower right corner of the right spectacle frame 103 and the lower left corner of the left spectacle frame 101, respectively, and there are an included angle d1 between one ultraviolet light source 20 and the right edge of the right spectacle frame 103, and an included angle d2 between another ultraviolet light source 20 and the left edge of the left spectacle frame 101. The included angle d1 and the included angle d2 can be any angle in the range of 10-60 degrees, such that the ultraviolet light source 20 can irradiate around, in front of, and above the oronasal position in the facial and surrounding area A1 of the user 60, so as to strengthen the disinfection and sterilization effect on the oronasal and surrounding position of the user 60.

In this embodiment, the ultraviolet light sources 20 are provided at the lower right corner of the right spectacle frame 103 and the lower left corner of the left spectacle frame 101, respectively. However, it should be noted that the ultraviolet light source 20 can be provided only at the lower left corner of the left spectacle frame 101 or the lower right corner of the right spectacle frame 103, or the ultraviolet light sources 20 can be provided both at the lower right corner of the right spectacle frame 103 and the lower left corner of the left spectacle frame 101, and it does not necessarily to have an included angle between the ultraviolet light source(s) 20 and the left and/or right edge. The disposing position of the ultraviolet light source(s) 20 at the lower left corner of the left spectacle frame 101 and/or the lower right corner of the right spectacle frame 103 can further prevent the irradiation path of the ultraviolet light from passing through the eyes.

### Sixth Embodiment

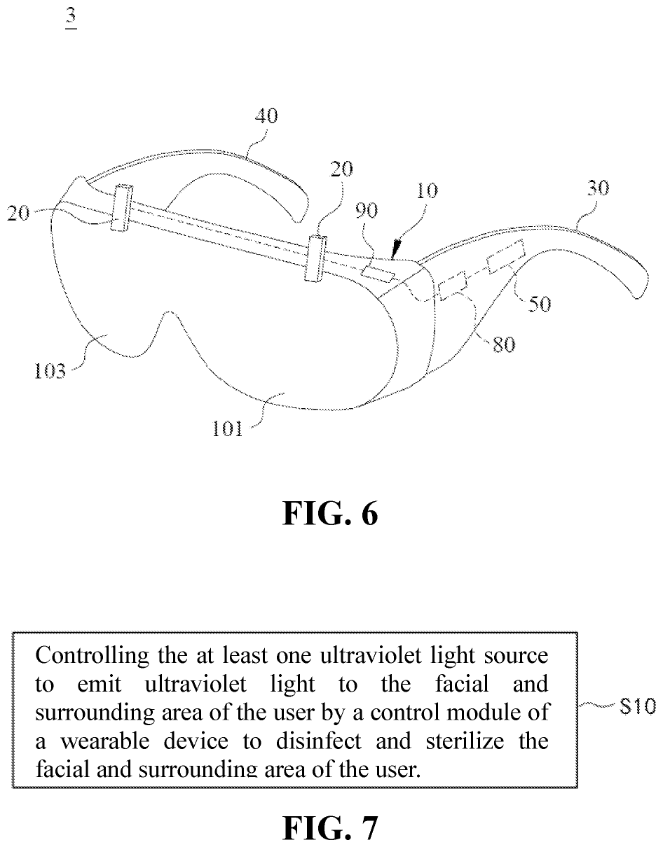

FIG. 6 is a schematic diagram illustrating a structure of a wearable device according to yet another embodiment of the present disclosure. Referring to FIG. 6, in this embodiment of the present disclosure, a wearable device 3 further comprises a control module 80 and/or a switch module 90. FIG. 6 shows that the control module 80 is provided in the left temple 30. However, it should be noted that the control module 80 can be selectively provided in the spectacle frame 10, the left temple 30, or the right temple 40. The control module 80 is electrically connected to the power supply device 50 and the ultraviolet light source 20, and the control module 80 controls the ultraviolet light source 20 to irradiate at a preset frequency. Wherein in this embodiment of the present disclosure, the preset frequency is any number of seconds in the range of 1/15 seconds to 1/30 seconds, which allows the ultraviolet light source 20 to form a pulse light source, such that the purpose of low power and power saving can be further achieved without losing the sterilization and disinfection effect. In addition, in this embodiment of the present disclosure, the control module 80 can control and adjust an irradiation angle of the ultraviolet light source 20, such that the ultraviolet light source 20 irradiates an oronasal and surrounding position in the facial and surrounding area of the user.

FIG. 6 shows that the switch module 90 is provided on the spectacle frame 10, but it should be noted that the switch module 90 can be selectively provided on the spectacle frame 10, the left temple 30, or the right temple 40, and the switch module 90 is electrically connected to the power supply device 50 and the ultraviolet light source 20, such that the turning on and off of the ultraviolet light source 20 can be controlled by the switch module 90, or whether the ultraviolet light source 20 is allowed to irradiate at the preset frequency can be further controlled by the switch module 90.

### Seventh Embodiment

FIG. 7 is a flow chart illustrating a method of using the wearable device according to an embodiment of the present disclosure. Referring to FIGS. 1, 3A, 3B and 7, in an embodiment of the present disclosure, when the wearable device has a control module 80 (as shown in FIG. 6), the method of using the wearable device comprises a step S10, in which: controlling the at least one ultraviolet light source 20 to emit ultraviolet light to the facial and surrounding area A1 of the user 60 by the control module 80 to disinfect and sterilize the facial and surrounding area A1 of the user 60, wherein the wearable device is worn on the ears 601 of the user 60 through the left temple 30 and the right temple 40, and is fixed to the nose 603 of the user 60 through the bridge 105.

On the other hand, referring to FIGS. 3B, 4 and 5 again, the method of using the wearable device of the present disclosure further comprises the following steps: controlling and adjusting an irradiation angle of the ultraviolet light source 20, such that the ultraviolet light source 20 irradiates an oronasal and surrounding position in the facial and surrounding area A1 of the user 60 by the user 60 or the control module 80, so as to strengthen the disinfection and sterilization effect on the oronasal and surrounding position of the user 60 and eliminate bacteria or droplets staying on the mask 70.

Furthermore, the user 60 or the control module 80 can further adjust the irradiation mode of the ultraviolet light source 20 such that the ultraviolet light source 20 can irradiate at a preset frequency. Wherein in the method of using the wearable device of the present disclosure, the preset frequency is any number of seconds in the range of 1/15 seconds to 1/30 seconds, which allows the ultraviolet light source 20 to form a pulse light source, such that the purpose of power saving can be further achieved on the premise of ensuring the sterilization and disinfection effect.

As can be seen from the above contents of the present disclosure, the present disclosure provides a wearable device and a method of using the same. The wearable device of the present disclosure can emit ultraviolet light to the facial and surrounding area of the user, and disinfect and sterilize the facial and surrounding area of the user through the ultraviolet light. Furthermore, when the user has worn the mask and covered the nose and mouth with the mask, the wearable device can emit ultraviolet light to disinfect and sterilize the mask and the area in front of the mask, eliminate bacteria attached to the mask, thereby allowing the user to rest assured. Therefore, the wearable device of the present disclosure not only can provide basic droplet blocking ability to protect the eye portion of the user, but also can further provide sterilization and disinfection effect on the facial and surrounding area of the user.

Furthermore, reference numerals and/or letters may be repeated in different examples in the present disclosure. This repetition is for purposes of simplicity and clarity and itself does not indicate the relationship between the various embodiments and/or arrangements discussed.

It should also be noted that, relational terms such as first and second used herein are only used to distinguish one entity or operation from another entity or operation, and do not necessarily require or imply that the existence of any such actual relationship or order between these entities or operations. Moreover, the terms “comprise”, “include” or any other variation thereof are intended to cover a non-exclusive inclusion.

These described above are optional embodiments of the present disclosure, it should be noted that a person skilled in the art can make several improvements and modifications without departing from the principles described in the present disclosure, these improvements and modifications should also be considered within the scope of the present disclosure.

What is claimed is:

1. A wearable device, comprising:
a spectacle frame including a left spectacle frame and a right spectacle frame, the left spectacle frame is connected to the right spectacle frame through a bridge;
at least one ultraviolet light source provided on the left spectacle frame and/or the right spectacle frame;
a left temple connected to the left spectacle frame;
a right temple connected to the right spectacle frame; and
a power supply device provided in the spectacle frame, the left temple, or the right temple, and the power supply device is electrically connected to the at least one ultraviolet light source, wherein the at least one ultraviolet light source emits ultraviolet light to a facial and surrounding area of a user.

2. The wearable device according to claim 1, further comprising a control module, the control module is provided in the spectacle frame, the left temple, or the right temple, and the control module is electrically connected to the power supply device and the at least one ultraviolet light source, the control module controls the at least one ultraviolet light source to irradiate at a preset frequency.

3. The wearable device according to claim 2, wherein the preset period is greater than or equal to 1/30 second and less than or equal to 1/15 second.

4. The wearable device according to claim 1, wherein the at least one ultraviolet light source is provided on a top surface of the left spectacle frame and/or a top surface of the right spectacle frame.

5. The wearable device according to claim 4, wherein there is an included angle between the at least one ultraviolet light source and the top surface of the left spectacle frame and/or the top surface of the right spectacle frame, such that the at least one ultraviolet light source irradiates an oronasal and surrounding position in the facial and surrounding area of the user, wherein the included angle is greater than or equal to 10 degrees and less than or equal to 60 degrees.

6. The wearable device according to claim 1, wherein the at least one ultraviolet light source is provided at a lower left corner of the left spectacle frame and/or a lower right corner of the right spectacle frame.

7. The wearable device according to claim 6, wherein there is an included angle between the at least one ultraviolet light source and a left edge of the left spectacle frame and/or a right edge of the right spectacle frame, such that the at least one ultraviolet light source irradiates an oronasal and surrounding position in the facial and surrounding area of the user, wherein the included angle is greater than or equal to 10 degrees and less than or equal to 60 degrees.

8. The wearable device according to claim 1, further comprising a switch module, the switch module is provided on the spectacle frame, the left temple, or the right temple, and the switch module is electrically connected to the power supply device and the at least one ultraviolet light source, the switch module controls a switching of the at least one ultraviolet light source.

9. The wearable device according to claim 1, wherein the at least one ultraviolet light source is at least one deep ultraviolet light source.

10. A method of using a wearable device, applied to the wearable device according to claim 1, in a case where the wearable device has a control module, the method comprises the following steps:
controlling the at least one ultraviolet light source to emit ultraviolet light to the facial and surrounding area of the user by the control module to disinfect and sterilize the facial and surrounding area of the user, wherein the wearable device is worn on ears of the user through the left temple and the right temple, and is fixed to a nose of the user through the bridge.

11. The method according to claim 10, further comprising the following steps:
controlling and adjusting an irradiation angle of the at least one ultraviolet light source by the control module, such that the at least one ultraviolet light source irradiates an oronasal and surrounding position in the facial and surrounding area of the user.

12. The method according to claim 10, wherein the control module controls the at least one ultraviolet light source to irradiate at a preset frequency.
